(19) United States
(12) Patent Application Publication
(10) Pub. No.: US 2022/0313315 A1
(43) Pub. Date: Oct. 6, 2022

(54) Morgellons Disease Diagnostic Device

(71) Applicant: Donna Roitstein, Quogue, NY (US)

(72) Inventor: Donna Roitstein, Quogue, NY (US)

(21) Appl. No.: 17/578,646

(22) Filed: Jan. 19, 2022

Related U.S. Application Data
(60) Provisional application No. 63/171,274, filed on Apr. 6, 2021.

(51) Int. Cl. A61B 17/52; G01N 37/00

(52) U.S. Cl. CPC A61B 17/52; G01N 37/005; A61B 2017/505

(57) ABSTRACT

The present invention relates to a Morgellons disease diagnostic device. The Morgellons disease diagnostic device features a round transparent glass bead attached to a simple hollow tube and is used along with an integrated or a coupled magnet. More specifically, the device uses magnetic means for absorbing microorganisms of a user or patient with Morgellons into the glass bead or similar device. Using the magnet, Morgellons parasites outer electrons gain energy which is dissipated as light allowing a user to view the parasites easily. The device and method provide a relatively safe, easy and convenient solution to identify and capture Morgellons disease causing pathogens.

### CROSS-REFERENCE TO RELATED APPLICATION

The present application claims priority to, and the benefit of, U.S. Provisional Application No. 63/171,274, which was filed on Apr. 6, 2021 and is incorporated herein by reference in its entirety.

### FIELD OF THE INVENTION

The present invention relates generally to a disease detector device. More specifically, the present invention relates to a Morgellons disease diagnostic device. The Morgellons disease diagnostic device of the present invention comprises a round transparent glass bead or the like, attached to a simple hollow tube used in conjunction with a magnet. The unique device uses magnetic means for attracting and absorbing microorganisms that may be in a patient with Morgellons parasites into the glass bead. Using the magnet, Morgellons parasites' outer electrons gain energy, which is dissipated as light, thereby allowing a user to view the parasites easily. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, Morgellons disease (MD) is a disorder characterized by the presence of fibers underneath, embedded in, and erupting from unbroken skin or slow-healing sores. MD is an emerging dermopathy with worldwide distribution. The name “Morgellons” is derived from a disease recognized in the seventeenth century in French children by Sir Thomas Browne. The most common symptoms of MD are the presence of small white, red, blue or black fibers under, on or erupting from sores or unbroken skin, causing the sensation that something is crawling on or under the skin.

In many cases, Lyme disease may be accompanied with Morgellons disease. The presence of Borrelia organisms and a spirochetal infection is generally associated with MD. In many clinical trials and studies, species of Borrelia including Borrelia burgdorferi, Borrelia garinii, Borrelia miyamotoi and Borrelia hermsii have been detected in skin specimens taken from MD patients.

Presently, a patient or individual cannot diagnose MD at home easily and must visit a doctor to diagnose MD. To diagnose MD, a doctor observes if a person has slow-healing skin lesions and skin-crawling sensations. If the doctor finds fiber-like material in the skin lesions, a sample can be sent to a laboratory for further analysis. The process takes a lot of time and in the times of pandemic, the patient or individual may not always be able to visit a doctor. Further, there is no specific cure for Morgellons disease, however, individuals who suffer from MD have been found to benefit from medications that treat psychosis or tics.

On many occasions, the doctor may not be able to correctly diagnose the MD. This can be extremely harmful for the patient and his or her caretaker. Also, treating MD can be difficult or nearly impossible, depending on several different factors. Thus, there is at least a need for an accurate and cost-effective approach and method for diagnosing MD for medical professionals and individuals at home.

Therefore, there exists a long felt need in the art for a Morgellons disease diagnostic device and method that offers a unique, inexpensive, and accurate method of identifying Morgellons disease-producing microbes. There is also a long felt need in the art for a Morgellons disease diagnostic device and method that can be used by both doctors and individuals at home. Additionally, there is a long felt need in the art for a Morgellons disease diagnostic device and method that captures the MD parasites easily. There is a long felt need in the art for Morgellons disease diagnostic device and method that illuminates the parasites, thereby allowing them to be observed easily for a considerable period of time. Further, there is a long felt need in the art for a Morgellons disease diagnostic device and method that can be used for capturing parasites from the skin and any exposed tissue easily and conveniently.

The subject matter disclosed and claimed herein, in one embodiment thereof, comprises a Morgellons disease diagnostic device. The diagnostic device captures microorganisms that can be found in an individual with Morgellons parasites. More specifically, the diagnostic device includes a transparent round glass bead, a hollow cylindrical tube and a magnet. The transparent round glass bead, or the like, is positioned on the top of the hollow cylindrical tube and captures Morgellons parasites and other co-infections.

The magnet excites outer orbital electrons of the parasites to create energy that is dissipated in the form of light, thereby allowing the images of the parasites to be visible with excellent resolution.

In this manner, the novel Morgellons disease diagnostic device and method of the present invention accomplishes all of the forgoing objectives, and provides a relatively safe, easy, and convenient solution to identify and capture Morgellons disease-causing pathogens. The MD device of the present invention is also user friendly, as it can be used by an individual, a patient, a caretaker or a medical professional. Additionally, the Morgellons disease diagnostic device can be used for any exposed skin tissue, along with the dermis, and for co-infections as well. Co-infection is the simultaneous infection of a host by multiple pathogen species, for instance multi-parasite infections. Co-infection also occurs as simultaneous infection of a single cell by two or more virus particles, which can arise incrementally from the initial infection to a superinfection.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a Morgellons disease diagnostic device. The diagnostic device is configured and designed to capture microorganisms that may be in an individual or patient with Morgellons parasites. The diagnostic device further comprises of a transparent round glass bead, a hollow cylindrical tube, and a magnet. The transparent round glass bead is positioned on the top of the hollow cylindrical tube and captures Morgellons parasites and other co-infections. The magnet excites outer orbital electrons of the parasites to create energy that is dissipated in the form of light, thereby allowing the images of the parasites to be visible with excellent resolution. The parasite and co-infection can escape from the round glass bead, and the round glass bead can be reused by the patient or user.

In a further embodiment of the present invention, a portable device for capturing Morgellons parasites and co-infections is disclosed. The device comprises: a hollow tube; a round transparent glass bead attached to the top of the hollow tube; a magnetic means of absorbing Morgellons parasites of an individual with Morgellons into the glass bead wherein the Morgellons parasites outer electrons gain energy; and, the energy is dissipated as light which allows a user to view the parasites in the light for observation.

In yet a further embodiment of the present invention, a method for capturing Morgellons parasites and other co-infections is described. The method comprises: initially, using a magnet to excite outer electrons of Morgellons parasites within a skin tissue area, under a dermis layer, or on a body part of a patient or individual; dissipating energy of the electrons in the form of light; capturing Morgellons parasites in a transparent rounded glass bead; and, observing movement of the parasites using the dissipated light. The method of the present invention offers a unique, inexpensive and accurate way of identifying Morgellons microorganisms and the extent of the spread of the disease.

In yet a further embodiment of the present invention, the detection device obviates the need for a user to use a magnifying glass or any topical gel to detect and diagnose Morgellons parasites and other co-infections. The parasites can be easily seen in the dissipated light and can be observed in motion for hours. Parasites from dermis, or any exposed tissue, can be easily detected using the method and device of the present invention. It is to be appreciated that the dermis layer is the layer of skin that lies beneath the epidermis and above the subcutaneous layer. It is the thickest layer of the skin and is made up of fibrous and elastic tissue.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a Morgellons disease diagnostic device of the present invention in accordance with the disclosed architecture;

FIG. 2 illustrates a perspective view showing a patient's or user's finger releasing Morgellons in the Morgellons disease diagnostic device of the present invention in accordance with the disclosed architecture; and

FIG. 3 illustrates a perspective view showing a co-infection or a parasite escaping from the detector glass bead of the Morgellons disease diagnostic device of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a Morgellons disease diagnostic device and method that offers a unique, inexpensive, and accurate method of identifying Morgellons disease-producing microbes. There is also a long felt need in the art for a Morgellons disease diagnostic device and method that can be used by doctors in a health care setting, and used by individuals/patients/users at home. Additionally, there is a long felt need in the art for a Morgellons disease diagnostic device and method that captures the MD parasites easily. There is a long felt need in the art for Morgellons disease diagnostic device and method that illuminates the parasites, thereby allowing them to be observed easily for a considerable period of time. Further, there is a long felt need in the art for a Morgellons disease diagnostic device and method that can be used for capturing parasites from skin and any exposed tissue easily and conveniently.

The present invention, in one exemplary embodiment, comprises a novel portable device for capturing Morgellons parasites and co-infections. The device includes a hollow tube, a round transparent glass bead attached to the top of the hollow tube and a magnetic means of exciting, attracting and absorbing Morgellons parasites of an individual with Morgellons into the glass bead. The Morgellons outer electrons gain energy, and the energy is subsequently dissipated as light which allows a user to view the parasites in the light for observation.

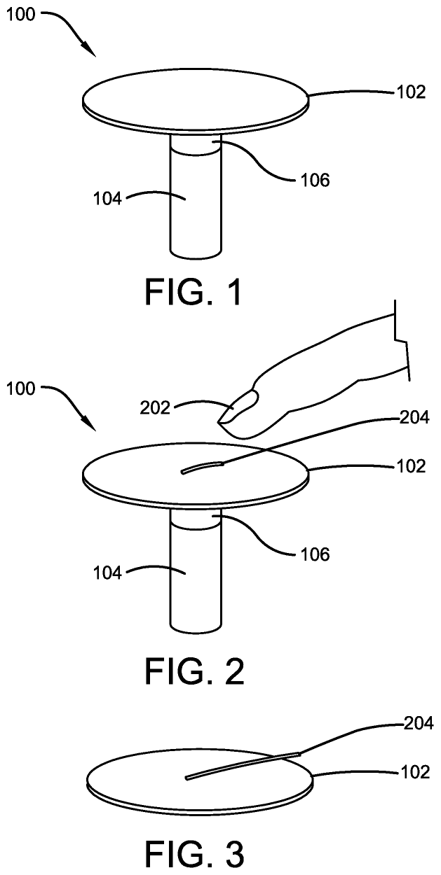

Referring initially to the drawings, FIG. 1 illustrates a perspective view of one potential embodiment of the Morgellons disease diagnostic device of the present invention in accordance with the disclosed architecture. The Morgellons disease diagnostic device 100 of the present invention is a handheld, portable device configured to quickly diagnose Morgellons disease and other co-infections. The device 100 includes a round transparent glass bead 102 at the top, that is attached to a hollow cylindrical tube 104. The glass bead 102 can comprise materials including a glass-filled polymer and glass-filled high density polyethylene (HDPE). The device 100 is used with a magnetic means 106 such as a permanent magnet for exciting parasites or other co-infection microbes of a patient, attracting the parasites and absorbing them into the round transparent glass bead 102.

During use, the magnetic means excites outer electrons of the Morgellons parasites. Energy of the parasites caused by the excited outer electrons is dissipated in the form of light, allowing the parasites to be clearly visible. The parasites, once illuminated, are absorbed by the round transparent glass bead 102. Due to the energy of the parasites, the parasites are seen in the dissipated light and can be observed in motion for hours, thereby allowing for easy capture by the glass bead 102. The device 100 along with the magnetic means 106 offers a unique, inexpensive and accurate method of identifying Morgellons disease-producing microorganisms and the extent of spread. The visually illuminated parasites are visible in the transparent round glass bead 102 and any co-infection can escape from the glass bead 102 into the hollow cylindrical tube 104, thereby separating Morgellons parasites from other co-infections.

FIG. 2 illustrates a perspective view showing an individual's finger releasing Morgellons in the Morgellons disease diagnostic device of the present invention in accordance with the disclosed architecture. When a Morgellons parasite 204 is excited using the magnet 106, the Morgellons parasite 204 crawling under user's skin exits the dermis. The dermis is the layer of skin that lies beneath the epidermis and above the subcutaneous layer. It is the thickest layer of the skin and is made up of fibrous and elastic tissue. Then, the parasite 204 is captured by the glass bead 102 when a body part or exposed tissue such as a finger 202 touches the transparent glass bead 102. The parasite 204 remains illuminated, due to energy of the outer electrons, for a few hours within the glass bead 102. The illuminated parasite 204 is easily visible in the glass bead 102 and can be used for research and testing for medication.

The magnet 106 can be permanently attached to the hollow tube 104 or can be removably attached to the tube 104. The magnet 106 can be used separately to excite the outer electrons of a parasite. It should be noted that the imaginary magnetic lines of force of the magnet 106 are distorted by the parasite 204, and thus cause illumination of the parasite 204. A small electromagnetic force is applied by the magnet on moving electrons of the parasite 204 for illumination.

The transparent glass bead 102 of the detector device 100 glows in the dark due to the illumination of the captured parasite. The detector device 100 is useful for capturing Morgellons parasites with co-infection as well. As per the preferences of a user, the magnet 106 can be selectively coupled or integrated to the detector device 100.

FIG. 3 illustrates a perspective view showing a co-infection or a parasite escaping from the detector glass of the Morgellons disease diagnostic device of the present invention in accordance with the disclosed architecture. A parasite or a co-infection 204 captured by the glass detector 102 can escape automatically or can be released by a user/patient. The glass detector 102 can be reused by an individual for capturing parasites and other co-infections. It is to be appreciated that co-infection is the simultaneous infection of a host by multiple pathogen species, for instance multi-e infections. Co-infection also occurs as simultaneous infection of a single cell by two or more virus particles, which can arise incrementally by the initial infection followed by a superinfection.

In one embodiment of the present invention, a method for capturing Morgellons parasites and other co-infections is described. The method comprises: using a magnet to excite outer electrons of Morgellons parasites within a skin tissue area, under a dermis layer or on a body part of a patient; attracting and pulling the Morgellons parasite from the skin tissue area or dermis layer; dissipating energy of the electrons in the form of light; capturing Morgellons parasites in a transparent rounded glass bead; and, observing movement of the parasites using the dissipated light. The method of the present invention offers a unique, inexpensive and accurate way of identifying Morgellons microorganisms and estimating the extent of spread of the disease.

It should be noted that the detector device 100 easily captures the Morgellons parasite and co-infections and obviates the need to use a microscope or magnifying glass to view or access the parasites. An individual/user/patient can see the parasites in light that results from the magnetic force. The parasites can be seen in excellent three-dimensional resolution in parallel light. Parallel light or a parallel light beam are light beams that are parallel to each other when coming from the source of light.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “Morgellons disease diagnostic device”, “detector device”, “device”, and “Morgellons device” are interchangeable and refer to the Morgellons disease diagnostic device 100 of the present invention.

Notwithstanding the forgoing, the Morgellons disease diagnostic device 100 of the present invention can be of any suitable size and configuration as is known in the art without affecting the overall concept of the invention, provided that it accomplishes the above-stated objectives. One of ordinary skill in the art will appreciate that the size, configuration, and material of the Morgellons disease diagnostic device 100 as shown in the FIGS. are for illustrative purposes only, and that many other sizes and shapes of the Morgellons disease diagnostic device 100 are well within the scope of the present disclosure. Although the dimensions of the Morgellons disease diagnostic device 100 are important design parameters for user convenience, the Morgellons disease diagnostic device 100 can be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disease detection device comprising:
a transparent glass bead;
a hollow tube; and
a magnet mounted between said glass bead and said hollow tube, wherein said magnet is positioned proximal to a user's skin to excite at least one outer electron of a parasite and attracts said parasite in a dermis layer of the user's skin.

2. The disease detection device of claim 1, wherein said transparent glass bead is a glass-filled polymer.

3. The disease detection device of claim 1, wherein said transparent glass bead is a glass-filled high density polyethylene.

4. The disease detection device of claim 1, wherein said parasite comprises a Morgellons parasite and said parasite is within a skin tissue area of a user.

5. The disease detection device of claim 1, wherein said parasite comprises a Morgellons parasite.

6. The disease detection device of claim 1, wherein said parasite comprises a Morgellons parasite and said parasite is on a body part of a user.

7. The disease detection device of claim 4, wherein said attraction of said parasite pulls said parasite from said skin tissue area of the user's skin.

8. The disease detection device of claim 7, wherein said transparent glass bead absorbs said parasite.

9. A method for capturing a Morgellons parasite, the method comprising the steps of:
holding a magnet next to a dermis layer of a user to excite at least one outer electron of the Morgellons parasite;
attracting the Morgellons parasite to the dermis layer;
pulling the Morgellons parasite from the dermis layer;
dissipating an energy of said outer electron in the form of a light;
capturing the Morgellons parasite in a transparent glass bead; and
observing movement of the Morgellons parasites using the light.

10. The method of claim 9 further comprising a step of identifying a Morgellons microorganism.

11. The method of claim 10 further comprising a step of estimating an extent of spread of a diseases caused by the Morgellons microorganism.

12. A method for detecting Morgellons disease and other co-infections, the method comprising the steps of:
providing a parasite capture device comprising a transparent glass bead, a hollow tube, and a magnet mounted between said transparent glass bead and said hollow tube;
positioning said magnet proximal to a user's skin to excite at least one outer electron of a Morgellons parasite;
attracting said Morgellons parasite in a dermis layer of the user's skin;
pulling said Morgellons parasite from the dermis layer;
dissipating an energy of said at least one outer electron in the form of a light;
capturing said Morgellons parasite in said transparent glass bead; and
observing movement of said Morgellons parasite using the light.

13. The method of claim 12, wherein said transparent glass bead is a glass-filled polymer.

14. The method of claim 12, wherein said transparent glass bead is a glass-filled high density polyethylene.

15. The device of claim 12, wherein said transparent glass bead absorbs said Morgellons parasite.

16. The method of claim 12 further comprising a step of identifying a Morgellons microorganism.

17. The method of claim 16 further comprising a step of estimating an extent of spread of the Morgellons disease.

18. The method of claim 16 further comprising a step of identifying the co-infections.

19. The method of claim 12, wherein said exciting outer electrons of said Morgellons parasite illuminates said Morgellons parasite.

20. The method of claim 18 further comprising a step of separating in said hollow tube said Morgellons parasite from the co-infections.
